(19) United States
(12) Patent Application Publication
(10) Pub. No.: US 2018/0261319 A1
(43) Pub. Date: Sep. 13, 2018

(54) NURSE SCHEDULING FORECASTS USING EMPIRICAL REGRESSION MODELING

(71) Applicants: Danielle Erin Bowie, Happy Valley, OR (US); Rachel Ann Fischer, Vancouver, WA (US)

(72) Inventors: Danielle Erin Bowie, Happy Valley, OR (US); Rachel Ann Fischer, Vancouver, WA (US)

(21) Appl. No.: 15/915,621

(22) Filed: Mar. 8, 2018

Related U.S. Application Data
(60) Provisional application No. 62/468,838, filed on Mar. 8, 2017.

(51) Int. Cl. G16H 40/20; G06N 99/00

(52) U.S. Cl. CPC G16H 40/20; G06N 99/005

(57) ABSTRACT

Nurse scheduling forecasts are enabled using empirical regression modeling. A regression model may be constructed for nurse scheduling. The nurse scheduling regression model may predict a number of nurses that need to be scheduled for various work shifts (e.g., night and day) for specified periods of time for various nurse specialties at one or more nursing facilities (e.g., an inpatient nursing unit). The model may be trained with historical data. Independent variables may include patient census at particular times of day and the number of nurses actually needed to provide patient care and/or comply with applicable policies and regulations, with the dependent variable being a prediction of the number of nurses to be scheduled in a specified period of time. Additional independent variables may include day of week, month of year, seasons and seasonal factors such as holidays and cultural events, staff vacations, and sick calls.

### CROSS-REFERENCES TO RELATED APPLICATIONS

This application claims the benefit of U.S. Provisional Application No. 62/468,838, filed Mar. 8, 2017, the entire contents of which is hereby incorporated in its entirety for all purposes.

### TECHNICAL FIELD

This invention relates generally to healthcare and more particularly to nurse scheduling.

### BACKGROUND

Historically, hospital nursing labor costs have been identified as a large contributor to overall healthcare cost. Over the years, many attempts have been made to contain and control nursing labor costs through a variety of scheduling tactics, but conventional efforts have shortcomings. Nurse leaders use multiple approaches when creating a schedule, but conventional data and decision support tools needed to create effective schedules are limited. This can result in schedules that cause a misalignment between the supply of nursing personnel and actual demand. Many hospitals use outdated methods for nurse scheduling, which can result in dramatic inefficiencies and high costs. Inadequate scheduling practices can result in substandard patient care. In addition, such practices can be a contributing factor to a poor work environment and even to health worker “burnout,” causing nurses to leave their jobs and even the profession, thereby contributing to industry wide shortages of skilled nurses.

Conventional attempts to address these issues are inefficient, ineffective and/or have undesirable side effects or other drawbacks with respect to at least one significant use case. For example, some conventional planning processes use a single averaged number representing patient census such as yearly averaged midnight patient census to determine daily resource needs. However, this leads to gross over and understaffing, a phenomenon known as “the flaws of averages.” Some conventional solutions are too narrowly focused, have technological support issues, have nursing acceptance issues, and/or are unable to incorporate particular management practices such as nurse shift self-allocation.

Embodiments of the invention are directed toward solving these and other problems individually and collectively.

### BRIEF DESCRIPTION OF THE DRAWINGS

Various embodiments in accordance with the present disclosure will be described with reference to the drawings, in which:

FIG. 1 is a schematic diagram depicting aspects of an example networking environment suited to at least one embodiment of the invention;

FIG. 2 is a schematic diagram depicting aspects of an example nurse scheduling forecast system in accordance with at least one embodiment of the invention;

FIG. 3 is a data flow diagram depicting aspects of an example nurse scheduling modeling process in accordance with at least one embodiment of the invention;

FIG. 4 is a graph depicting aspects of example nurse scheduling data in accordance with at least one embodiment of the invention;

FIG. 5 is a graph depicting further aspects of example nurse scheduling data in accordance with at least one embodiment of the invention;

FIG. 6 is a schematic diagram depicting aspects of an example nurse scheduling user interface in accordance with at least one embodiment of the invention;

FIG. 7 is a schematic diagram depicting further aspects of an example nurse scheduling user interface in accordance with at least one embodiment of the invention;

FIG. 8 is a flowchart depicting aspects of an example procedure for nurse scheduling forecasting in accordance with at least one embodiment of the invention; and

FIG. 9 is a schematic diagram depicting aspects of an example computer in accordance with at least one embodiment of the invention.

### DETAILED DESCRIPTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

In accordance with at least one embodiment of the invention, nurse scheduling forecasts are enabled using empirical regression modeling. For example, a linear regression model or an autoregressive integrated moving average (ARIMA) model may be constructed for nurse scheduling. The nurse scheduling model may predict a number of nurses that need to be scheduled for various work shifts (e.g., night and day) for specified periods of time for various nurse specialties at one or more nursing facilities (e.g., an inpatient nursing unit). The model may be trained with historical data. Core independent (input or data) variables may include patient census at particular times of day (e.g., 1700 and 0500, expressed in 24 hour time) and the number of nurses actually needed to provide patient care and/or comply with applicable policies and regulations, with the dependent (output or forecast) variable being a prediction of the number of nurses to be scheduled in a specified period of time (e.g., per 12-hour shift). Secondary independent variables may include (but are not limited to) day of week, month of year, seasons and seasonal factors such as holidays and cultural events, staff vacations, nurse sick calls, demographic trends (e.g., regional population and age trends), and seasonal disease intensity (e.g., influenza outbreak level).

Suitable historical data may include scheduling and staffing information for various nursing units such as medical-surgical, pediatric, critical care, intermediate care, and women's specialty units. Data points in the staffing datasets may include: the number of scheduled nurses, the day of staffing need for nurses, number of resource nurses used, sick calls, vacation, family medical leave act (FMLA) absences, nurses cancelled, overtime, premium pay shifts, nurses floated in and out of the unit, sitters, resource nurse, unfilled shifts, travelers (e.g., 3^{rd} party or “agency” nurses), patient census, patient admissions, patient discharges, patient length of stay (LOS), scheduled surgeries or other significant treatments, patient acuity (e.g., severity or type of illness), nurse skill set (sometimes called “nurse type”), and geographical distribution of nurses among nursing units. That is, these types of data points may be independent variables of the regression model. These independent variables may themselves be modeled and estimated for future time periods, for example, based on type of shift, shift start time, shift end time, day of week, month of year, seasons and seasonal factors such as holidays, sporting events and cultural events (collectively “time interval type”). Independent variables may be estimated utilizing any suitable estimation technique, including conventional estimation techniques. Alternatively, or in addition, independent variables for a future time interval may be estimated based only or substantially on time interval types occurring during the future time interval.

Autoregressive integrated moving average (ARIMA) modeling may be utilized. Alternatively, or in addition, other regression models such as linear regression models and generalizations of autoregressive moving average (ARMA) models may be utilized including seasonal ARIMA models, nonlinear autoregressive moving average (NARMA) models, autoregressive conditional heteroskedasticity (ARCH) models, autoregressive fractionally integrated moving average (ARFIMA or FARIMA) models, autoregressive moving average with exogenous inputs (ARMAX) models, and any suitable time series analysis techniques for modeling a stationary stochastic process.

A goal of a manager in making a schedule is to ensure “the assignment of the right people to the right task, to the right time and to the right place.” The schedule problem for nurses (the “nurse scheduling problem”) has not been solved. It involves multifaceted operative management that does not operate from a simple supply-and-demand economic model. Rather there are multiple suggestions and philosophies on how to achieve appropriate nurse scheduling.

When creating a schedule, nurse leaders manually consider a variety of weighted variables during the building phase of the scheduling process. Examples of variables include federal and state regulations, patient characteristics, nurse characteristics, shift length, geographical layout of the nursing unit, technology, cost, supply, and theoretical models of staffing. Once a schedule has been built, many leaders spend a significant amount of administrative time managing and maintaining the published schedule. The majority of that time is spent reworking and changing the schedule to accommodate for unexpected variables such as emergency leave, sickness, study days, worked overtime and fluctuations in bed occupancy.

Basic staffing and scheduling methods of the past are not meeting the complex demands of the present and future staffing environment for nursing. The chaotic and changing environment of healthcare requires a more sophisticated supply-and-demand simulation modeling to predict and plan for nurse staffing needs. Planning methods must evolve into more sophisticated forecasting modeling techniques that support nurse leaders in meeting the increasing demands of personnel scheduling needs.

The nurse staffing process may be divided into three phases. The first phase of the nurse staffing process is budgeting and/or planning (sometimes called “requirement” or “recruitment”). This stage is the long-range planning of personnel skill mix, specifying the number of annual full-time equivalents (FTEs) needed over several months (e.g., 6 to 18 months). The second phase is scheduling, the creation of a multi-week schedule (e.g., 4 to 8 weeks), determining when nurses will and will not work. The third phase is called staffing or allocation and deals with the real-time or “day of” distribution of nurses to handle actual workload (sometimes called “re-allocation”). This third phase is reactive in contrast to the goal setting of the second phase. For example, abnormal numbers of admissions, discharges, and/or nurse absence may require adjustments in the third phase. The three phases of the nurse staffing process are interrelated, and each phase impacts the overall outcomes associated with nurse staffing. Conventional attempts to solve the three phases of the nurse staffing process simultaneously has been found to be infeasible due to the complexity of the problem.

The nurse scheduling problem may be divided into six modules that can be solved and implemented sequentially or combined depending on the build of the nursing schedule: demand modeling, day off scheduling, shift scheduling, line of work construction, task assignment, and staff assignment. The first step, demand modeling, translates predicted patterns of incidents to determine the demand for staff. Additionally, demand modeling may have three subsequent categories: task-based demand, flexible demand, and shift-based demands. Shift-based demand, in particular, is often associated with nurse scheduling because the demand of “staff levels are determined by a need to meet services measures such as nurse/patient ratios.” Due to the computational complexity of the nurse scheduling problem, the step of demand modeling to predict patterns and forecast schedule needs may need to be evaluated and solved as a separate module.

The demand modeling phase may translate empirical incident data to a demand for staff, and a method for forecasting incidents. In the hospital setting, the number of nurses needed may be related to the number of patients and organizational or state regulations concerning nurse-to-patient ratios. However, this is not the whole story. Factors other than patient admissions and discharge can have significant influences on nurse demand, for example, selected sets of the independent variables described herein. Different approaches exist for forecasting the distribution of incident data for staff demand over a planning horizon. Those approaches include simple averaging, exponential smoothing, and regression modeling including linear regression models as well as seasonal and nonseasonal autoregressive integrated moving average (ARIMA) models.

In the “Box-Jenkins” approach to ARIMA modeling, an ARIMA model is built using an iterative process that includes ARIMA model class identification, unknown parameter estimation, and diagnostic checks to determine the model adequacy and fit. Model identification is the process of evaluating the source of the data and the collection methods. For data evaluation, plotting the data with methods such as simple time-series or scatter plots is necessary and helpful to determine stationarity vs. nonstationarity, which is the extent the data shows similarity over time. Final evaluation of the data may include autocorrelation coefficient (ACF) and partial correlation coefficient (PACF) measures. The process of parameter estimation may be performed using statistical analysis software (e.g., SAS®). Diagnostic checking includes statistical tests such as chi-square, the degree of freedom, and the Ljung-Box test. Finally, validation of model fit includes common measures such as mean absolute percentage error (MAPE) and root mean square average (RMSE), which may be used with Akaike's Information Criterion (AIC) and Schwartz's Bayesian Criterion (SBC).

In accordance with at least one embodiment of the invention, a regression model such as a linear regression model or an ARIMA model is constructed to guide personnel resource planning that forecasts the number of nurses that need to be scheduled on any given day or shift. This is in contrast to regression models that forecast different variables such as those associated with volume metrics, for example: outpatient visits, surgical volumes, emergency department patient visits, the number of bed days, patient volumes, patient length of stay, ICU admissions, and budgeted nursing hours. There may be a variety of forecasted time increments for the forecasted parameter including hourly, daily, weekly, monthly and yearly forecasts. Models may include a variety of independent variables related to external factors such as patient characteristics, types of surgery, physician staffing, resuscitation cases, meteorological measurements, holidays, and surgical times.

In addition, the regression forecasting method may be paired with optimization models in a two-staged approach. For example, the regression model may be used to predict the number of nurses that need to be scheduled, and an optimization model or queuing methods may be used to allocate nurses to meet scheduled number of nurses for a particular time period. Note that the regression modeling sets goal numbers, while the staffing optimization process allocates staff from various skill pools to meet those goals.

In accordance with at least one embodiment of the invention, a generalized nurse scheduling regression model is developed and implemented based on daily organizational scheduling and staffing data. The model predicts the daily number of nurses that need to be scheduled for various shifts (e.g., day shifts and night shifts) in a multi-week (e.g., 6 week) schedule cycle for various patient care units including critical care, medical-surgical, intermediate care, pediatrics, and labor and delivery inpatient units. In accordance with at least one embodiment of the invention, the nurse scheduling regression model produces better nurse schedule management, thereby reducing a rate of unfilled shifts, premium paid shifts, and overtime experienced by inpatient nursing units (collectively, “schedule variances”). In particular, the nurse scheduling regression model may benefit by selecting particular sets of independent variables. For example, by utilizing particular patient census times (e.g., 0500 and 1700) rather than others (e.g., at midnight). Alternatively, or in addition, the nurse scheduling ARIMA model may reduce schedule variances by varying shift start and/or end times.

Pre-existing electronic scheduling systems may be adapted to use forecasts generated by the nurse scheduling regression model. For example, by adjusting a simple average-based number of nurses with the more sophisticated forecast numbers in the electronic scheduling system. In accordance with at least one embodiment of the invention, utilizing regression model forecasts for nurse scheduling results in a reduction in a rate of unfilled nursing shifts and overtime nursing shifts experienced by inpatient nursing units, which can yield significant cost savings.

Historical, empirical data may be by any suitable time period including: year, season, month, day of the week, shift (e.g., day, night), and inpatient unit.

There follows descriptions of some nursing-related terms as used herein. Although, for clarity, nurses are used as an example throughout this application, the systems and methods described herein may be applied to any suitable healthcare worker or provider.

Number of Nurses Need Day of Staffing: The number of nurses needed to staff an nursing unit (e.g., an inpatient unit) for an immediately pending shift (e.g., an upcoming 12-hour shift). For example, the charge nurse may determine the immediate staffing need for the nursing unit. For example, the determination for a 12-hour day shift may be made at 0500 and at 1700 for a 12-hour night shift.

Resource Nurse: A “float” nurse who is assigned to a unit to meet the day of staffing need and who is not pre-scheduled to a nursing unit.

Number of Nurses Floated: The number of nurses that are either floated in or out of an nursing unit. When there is a surplus of nurses on a unit, a nurse will be floated to a similar unit if that unit has a staffing need. Similarly, when an inpatient unit does not have enough nurses, a nurse is floated to that unit from an another unit when the other unit has a surplus of nurses, and there is no staffing need.

Number of Nurses Cancelled: A nurse may be canceled for his or her shift when there are more nurses scheduled than are needed, and there is no staffing need for the nurse to float to another unit.

Travelers: Contract labor nurses known as travelers may be hired for short-term (e.g., 13-week) assignments from 3^{rd} party staffing agencies. Traveler nurses are not employees of the organization they are working for and are typically paid at a higher hourly rate. The contract labor nurse may be assigned and scheduled to a unit or group of units for the term of the contract.

Family and Medical Leave Act: A protected leave of absence, which could be intermittent or continuous.

Premium pay shifts, paid at a multiple (e.g., 190%) of the nominal hourly rate.

Overtime: A shift allocated due to understaffing, also paid at a multiple (e.g., 150%) of the nominal hourly rate.

The description now turns to the Figures, which illustrate aspects of the discussion above.

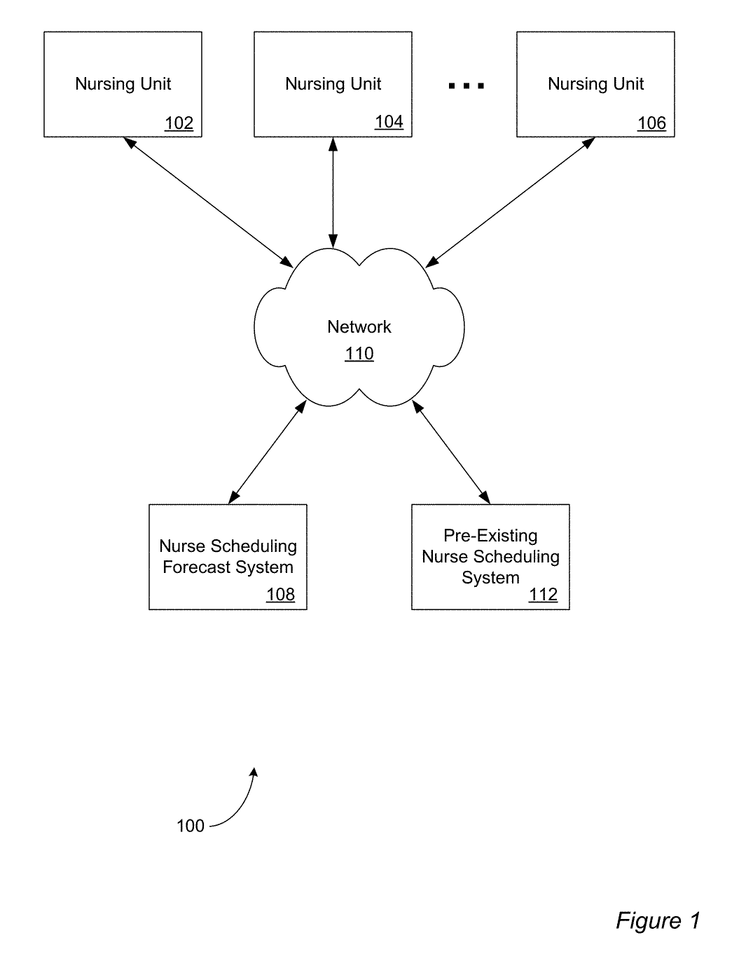

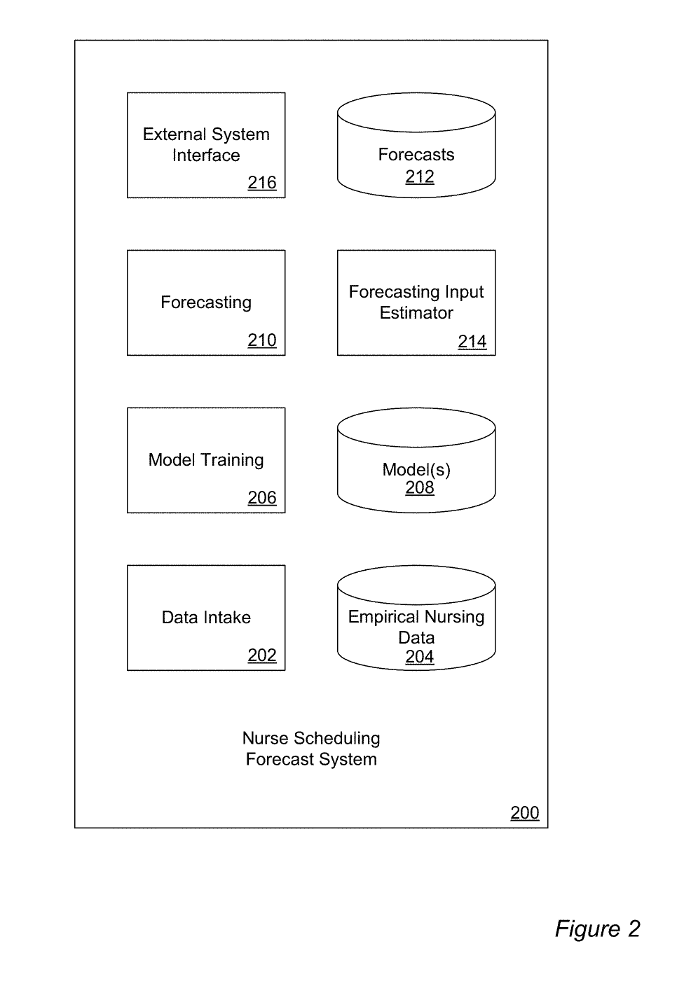

FIG. 1 depicts an example networking environment 100 suited to at least one embodiment of the invention. Multiple nursing units 102, 104, 106 may provide data (e.g., empirical nursing data such as staffing datasets) to a nurse scheduling forecast system 108 over a network 110. The nurse scheduling forecast system (described below in more detail with reference to FIG. 2) may generate nurse scheduling forecasts and provide them to a pre-existing nurse scheduling system 112 (e.g., an established and/or conventional nurse scheduling system utilizing simplistic nurse schedule setting). The pre-existing nurse scheduling system may then update its scheduling goals with the forecasts provided by the nurse scheduling forecast system and provide nurse scheduling and/or staffing services as usual. In an alternate example, the nurse scheduling forecast system may be integrated with the pre-existing nurse scheduling system.

The nursing units may be at disparate geographical locations and may utilize computer systems at those locations to communicate over the network with the nurse scheduling forecast system and the pre-existing nurse scheduling system. The network may incorporate any suitable networking technology including wired and wireless networking technologies. Although multiple nursing units are depicted, a single nursing unit may be utilized in accordance with at least one embodiment of the invention. Alternatively, or in addition, a serverless networking environment (sometimes called a peer-to-peer or overlay networking environment) may be utilized in accordance with at least one embodiment of the invention. Where functionality is divided between a client and a server, some or all of the functionality may be relocated from the client to the server (e.g., with “thin” client techniques). Alternatively, some or all of the functionality may be relocated from the server to the client (e.g., with “fat” client techniques and/or serverless networking technologies). The distribution of functionality between client and server may be fluid and adaptive (e.g., to client, server and/or network performance). Client-server may be poll driven (e.g., servers are relatively passive, responding to client “polls” or requests) and/or event driven (e.g., servers actively “push” events to interested or subscribed clients). In FIG. 1, and throughout this specification, the ellipsis is used, as is conventional, to indicate “any suitable number” of objects.

FIG. 2 depicts an example nurse scheduling forecast system 200 in accordance with at least one embodiment of the invention. A data intake module 202 may receive empirical nursing data 204 from the nursing units, parse, “clean” and otherwise process the data, and store the data in a database. A model training module 206 may train a regression module utilizing the empirical nursing data to arrive at a model 208. There may be multiple models, for example, a model for each nursing unit and/or type of nursing. A forecasting module 210 may select an appropriate model for a forecast and generate a nurse scheduling forecast 212. Where particular independent variables require estimation for particular future time intervals, a forecasting input estimator module 214 may generate one or more estimates for each time interval (e.g., the model training module may train models for such parameters, or more simplistic estimations rules may be configured). An external system interface module 216 may manage interaction with computer systems at the nursing units and/or the pre-existing nurse scheduling system. In some examples, multiple such external system interface modules may be required, e.g., one per type of external system.

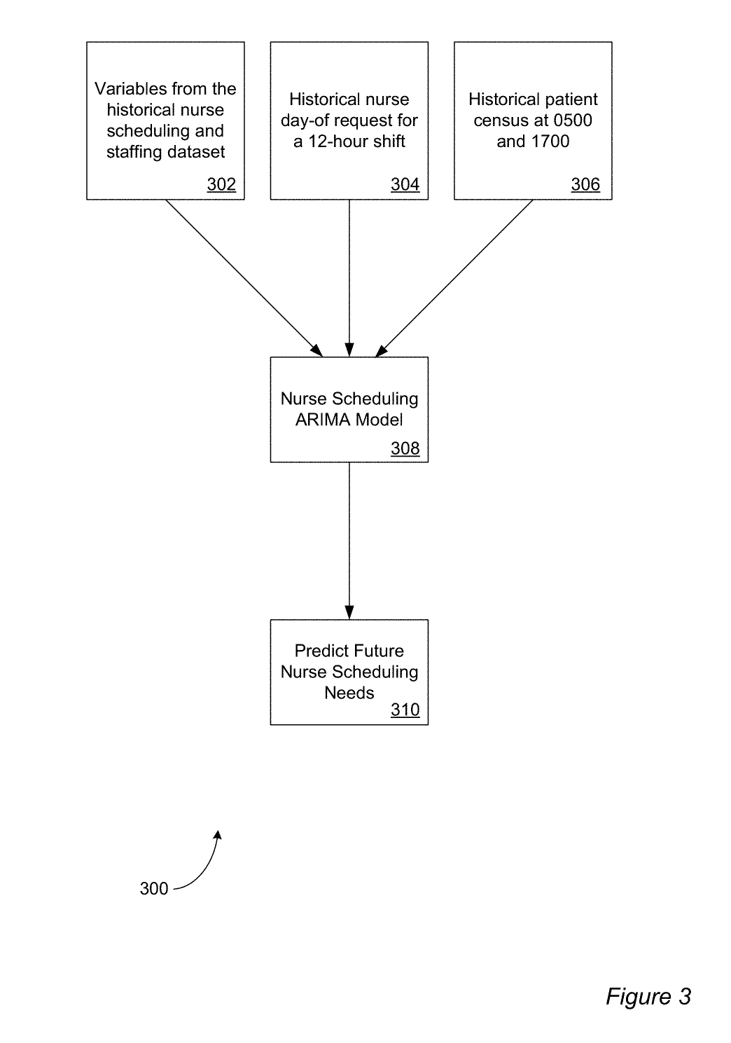

FIG. 3 depicts an example nurse scheduling modeling process 300 in accordance with at least one embodiment of the invention. In this example, an ARIMA model 308 is trained utilizing variables from an historical nurse scheduling and staffing dataset 302, historical nurse day-of-request for a 12-hour shift (e.g., actual nurses needed at time of staffing) 304, and historical patient census at 0500 and 1700 (specified in 24 hour time) 306. Historical data may be by year, season, month, day of the week, shift (day/night), and inpatient unit. The trained ARIMA model may then be utilized to forecast future nurse scheduling needs 310 for various time periods, e.g., for a season, a month, a day of week, a shift type, and for given nursing units. For example, the forecast nurse scheduling may include a number of nurses to be scheduled for the future time interval such that a difference between the number of nurses to be scheduled and an estimated actual nurse demand during the future time interval is optimized (e.g., minimized).

In another example nurse scheduling modeling process in accordance with at least one embodiment of the invention, linear regression may be used to predict the impact of the number of nurses as a function of the patient census. Utilizing data obtained from an inner-city urban hospital including nurse staffing, scheduling, and patient census data for a 33-bed surgical specialties unit, a bivariate linear regression model may be estimated, for example:

Number of nurses needed=2.96+0.168*(Census count)

Once the nurse-to-census relationship is established, a census forecast may be utilized to make the nurse-needed projection for a scheduling periods, such as a six-week scheduling period. An adjusted simple average of the previous time period of the 0300 census may be utilized. The census forecast may be created by taking the average of the last two years of that same six-week schedule period (e.g., averaging over the six week period) and shifting the date to align by day of the week, for example, resulting in the following:

$$Census forecast = ( \frac{( {2015}_{date by day of week} + {2016}_{date by day of week} )}{2} + 3 )$$

In this example, the simple average is adjusted for local patient census growth (e.g., responsive to demographic change). The three (3) units were added to the census because on average, the patient census for 2017 was three patients higher than for the two previous years. Additionally, the data showed a variation in the day of the week for the nurse-needed, with a consistent weekly trend of more nurses needed Tuesday through Friday and fewer nurses needed

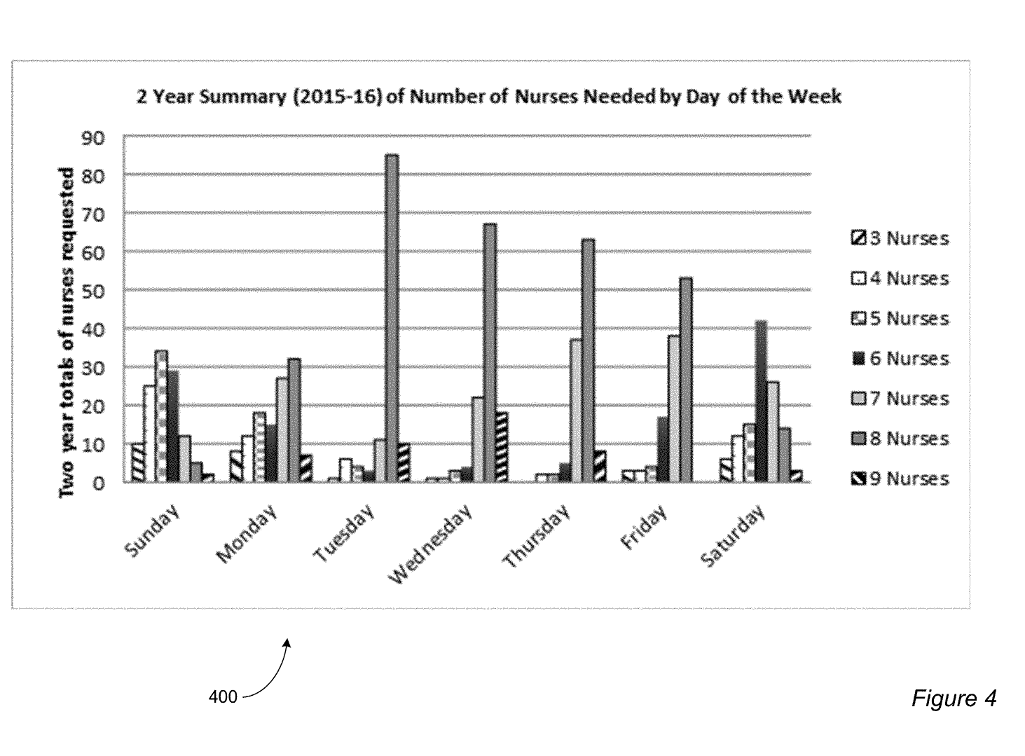

Saturday through Monday. FIG. 4 is a graph 400 showing example data including the frequency of nurses needed by day of the week for 2015 and 2016. For example, on Tuesdays 8 nurses were needed for the day shift 83 times and 4 nurses were needed only 5 times. Alternatively, or in addition to the above adjusted simple average technique, more sophisticated modeling may be utilized. For example, a non-linear regression may be employed to determine parameter values (A, B, C, D) of the following expression:

f(x)=A sin B(x−C)²+D

where f(x) is the census forecast for the day number x in the scheduling period. More sophisticated patient census modeling may enable more accurate patient census forecasts.

In this example, a six-week scheduling cycle was used for the testing period of Nov. 12, 2017 to Dec. 23, 2017. Eight weeks before the beginning of the test period, the nurse scheduling needs for the day shift were adjusted in the pre-existing electronic scheduling system to match the predicted needs generated from the model. The implementation of model predictions was made before the employee self-scheduling period. After predictions were implemented, unit scheduling practices of employee self-scheduling and managerial balancing resumed for the creation and publishing of the six-week schedule.

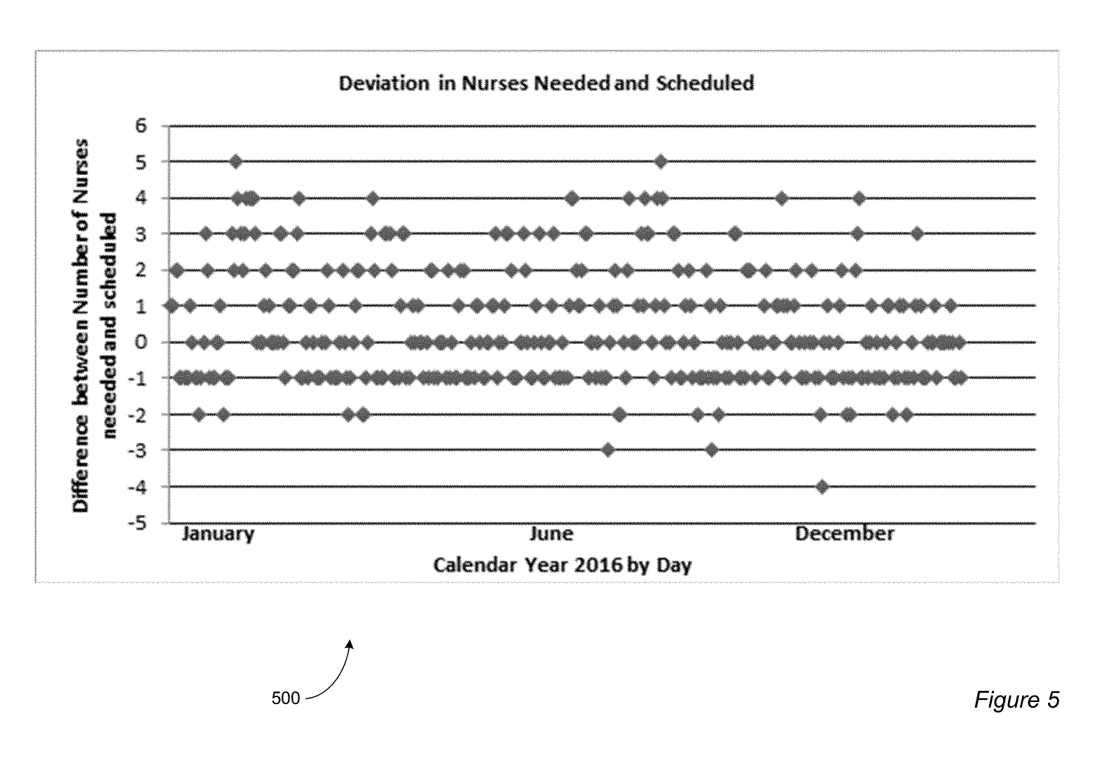

In this example, analysis of the predictions were compared to past scheduling practices, revealing that current and past scheduling practices were consistently underestimating by 1.2 nurses the number of 12-hour dayshift nurses needed. Additionally, there were wide deviations of nurse schedule needs ranging from underpredicting by 4 nurses and overpredicting by 5 nurses per shift as illustrated by the graph 500 of FIG. 5. In the graph 500, a y-axis value of 0 indicates that staffing was optimal, positive values indicate overstaffing and negative value indicate understaffing. In accordance with at least one embodiment of the invention, the forecast model resulted in more accurate scheduling predictions by reducing the wide variations associated with past scheduling practices. For example, a reduction in error between forecast and actual demand can be beneficial. Such error values may be the absolute value of the y-axis in FIG. 5. Alternatively, the forecast may seek to reduce mean squared error or any suitable loss function. Alternatively, overstaffing may be treated differently from understaffing (e.g., the loss function need not be symmetrical).

In this example, in addition to underestimating dayshift nurse requirements, the old scheduling methods and practices for this inpatient unit did not account for variation in the number of nurses needed by day of the week. In accordance with at least one embodiment of the invention, the forecast model, however, predicted weekly variation: more nurses needed from Tuesday through Friday, with a gradual tapering off and fewer nurses needed from Saturday through Monday.

In accordance with at least one embodiment of the invention, it is better in practice to over-predict than to under-predict the number of nurses needed. If an organization has scheduled too many nurses, it is possible for one or more nurses to be canceled or placed on standby. Under-predicting can mean that there is no nurse to care for an actual patient and the shift was either unfilled, with fewer nurses (e.g., a suboptimal number of nurses) caring for the patients, or a premium paid shift was offered as an incentive to get one or more nurses to come in to work, resulting in higher staffing costs. Days at zero represent accurate prediction. The following table provides some illustrative statistics for this example:

| | 2017 Predicted Dayshift RN | 2016 Predicted Dayshift RN |
|---|---|---|
| | vs. Actual Dayshift Needed - | vs. Actual Dayshift Needed - |
| 6 Weeks Schedule Cycle: 42 | Live Prediction | Training Period |
| days | (Nov. 12, 2017-Dec. 23, 2017) | (Nov. 12, 2016-Dec. 23, 2016) |
| Average Deviation | 0.20 | −0.71 |
| Number of days at zero | 22 | 13 |
| Number of days under | 7 | 26 |
| prediction | | |
| Number of days over | 13 | 3 |
| prediction | | |

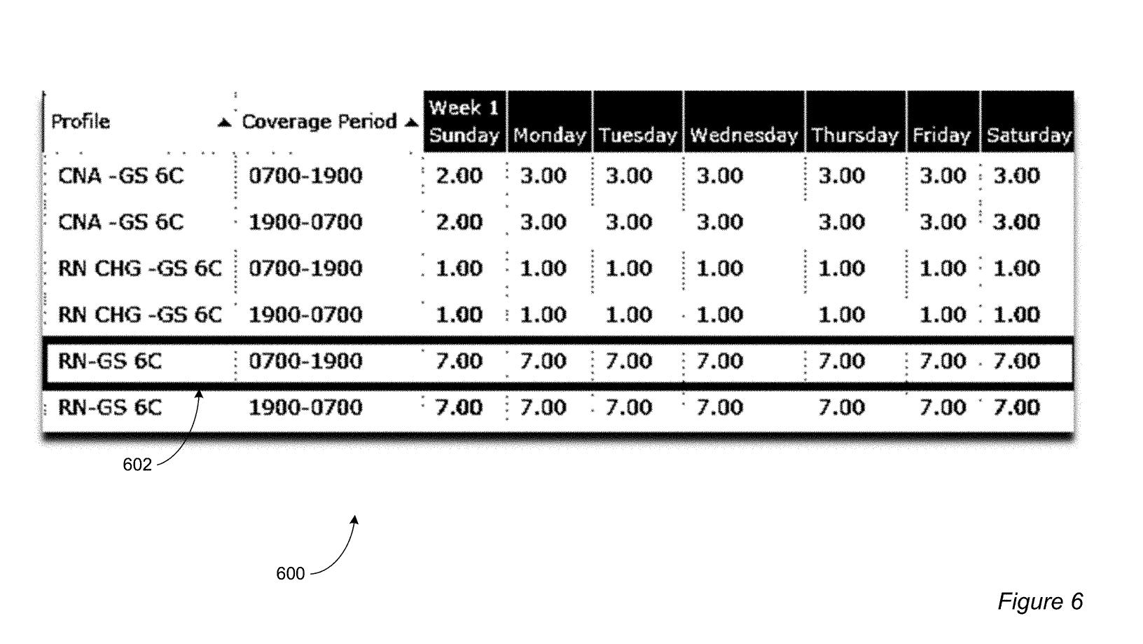

FIG. 6 is an example user interface 600 showing an example one-week schedule template that specifies the total number of dayshift nursing needs before model predictions were conducted. The template indicates a need for one 0700-1900 charge nurse (RN CHG-GS 6C) and seven direct care nurses (RN-GS 6C) daily, totaling eight nurses to be scheduled every day. This same template was applied to every week of the year in the pre-existing nurse scheduling system.

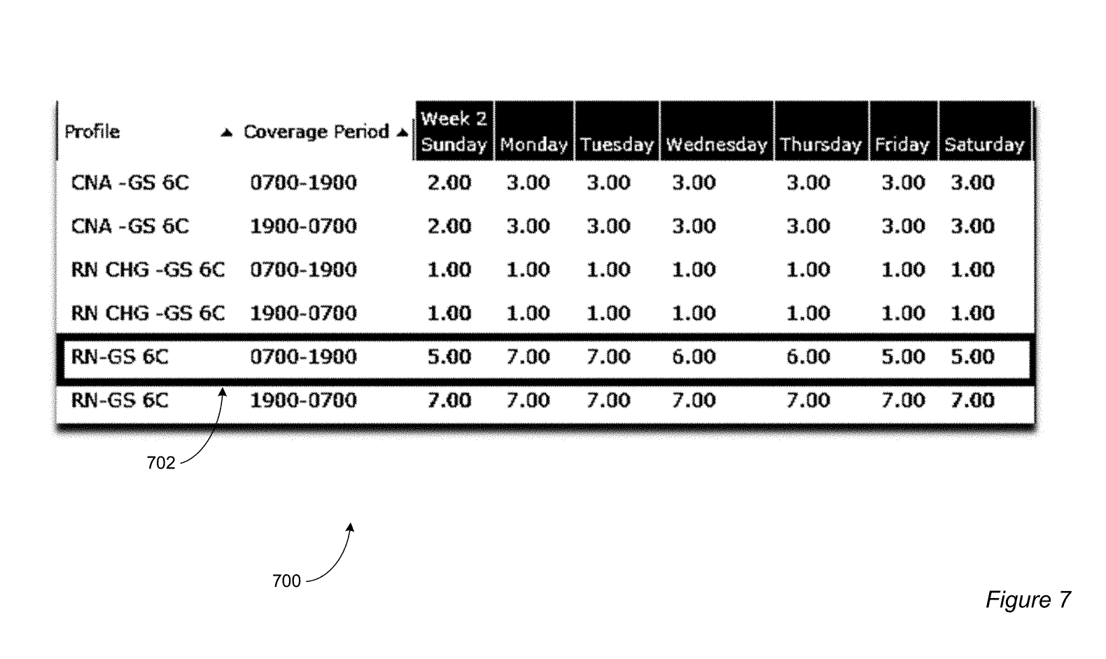

FIG. 7 is an example user interface 700 showing how the staffing template may be updated to reflect model predictions for day shift nurses that accounted for variation on the day of the week. The highlighted shift 702 (RN-GS 6C, 0700-1900) has updated staffing with respect to the same shift 602 shown in FIG. 6. In accordance with at least one embodiment, each week of the six-week schedule may have a different template to reflect the predictions associated with that period.

In this example, the unit's past scheduling practices had been derived from hour-per-patient day values calculated from a yearly averaged midnight patient census. This practice was perpetuating the “flaws of average” phenomenon for scheduling, which in turn led to daily over-or understaffing. In this example, a regression model used historical daily patient census data at 0300 to provide more relevant knowledge for accurate schedule predictions for the 0700-day shift, thus reducing over- or understaffing.

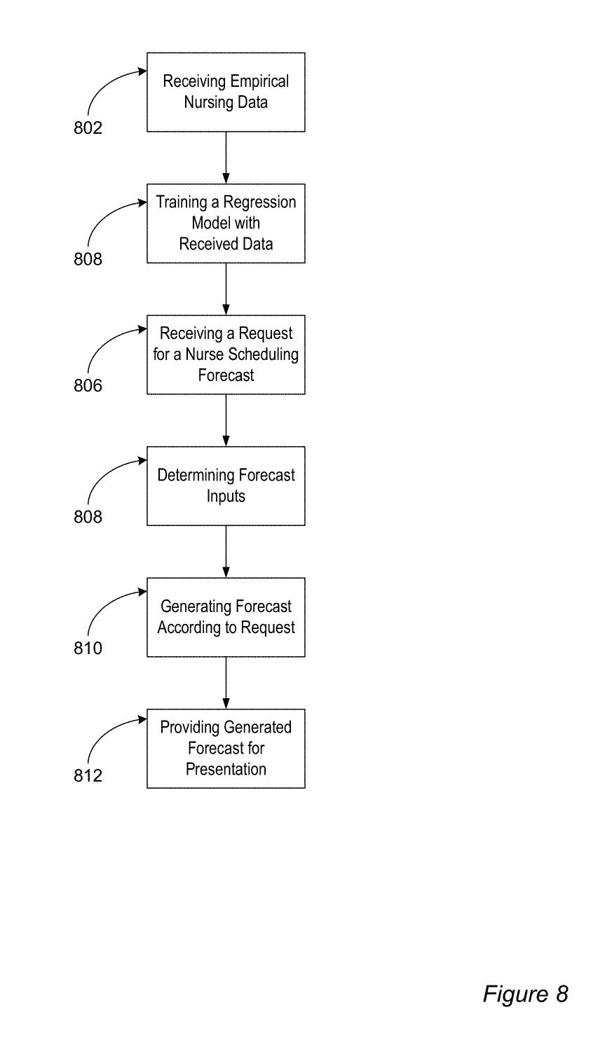

FIG. 8 depicts operations of an example procedure for nurse scheduling forecasting in accordance with at least one embodiment of the invention. At 802, empirical nursing data may be received, for example, from operational nursing units 102, 104, 106 (FIG. 1). At 804, a regression model such as a linear regression model and/or an ARIMA model may be trained with the data received at 802, for example, by the nurse scheduling forecast system 108. At 806, a request may be received for a nurse scheduling forecast. For example, the pre-existing nurse scheduling system 112 may send a request to the nurse scheduling forecast system specifying a future time interval, as well as other independent variables for the forecast if any. At 808, forecast inputs may be determined. For example, it may be that all required forecast inputs have been received in the request of 806. Alternatively, it may be that one or more required forecast inputs should be estimated and/or derived from received data. For example, the forecasting input estimator module 214 of FIG. 2 may determine one or more of these forecast inputs (including by utilizing regression models such as linear and/or ARIMA models for such parameters). At 810, a nurse scheduling forecast may be generated in accordance with the request of 806, for example, by the forecasting module 210 of FIG. 2. At 812, the generated forecast may be provided for presentation to a user. For example, the generated forecast may be provided to the pre-existing nurse scheduling system for use in updating scheduling goals. Alternatively, or in addition, the generated nurse scheduling forecast may be provided for presentation to users at the nursing units.

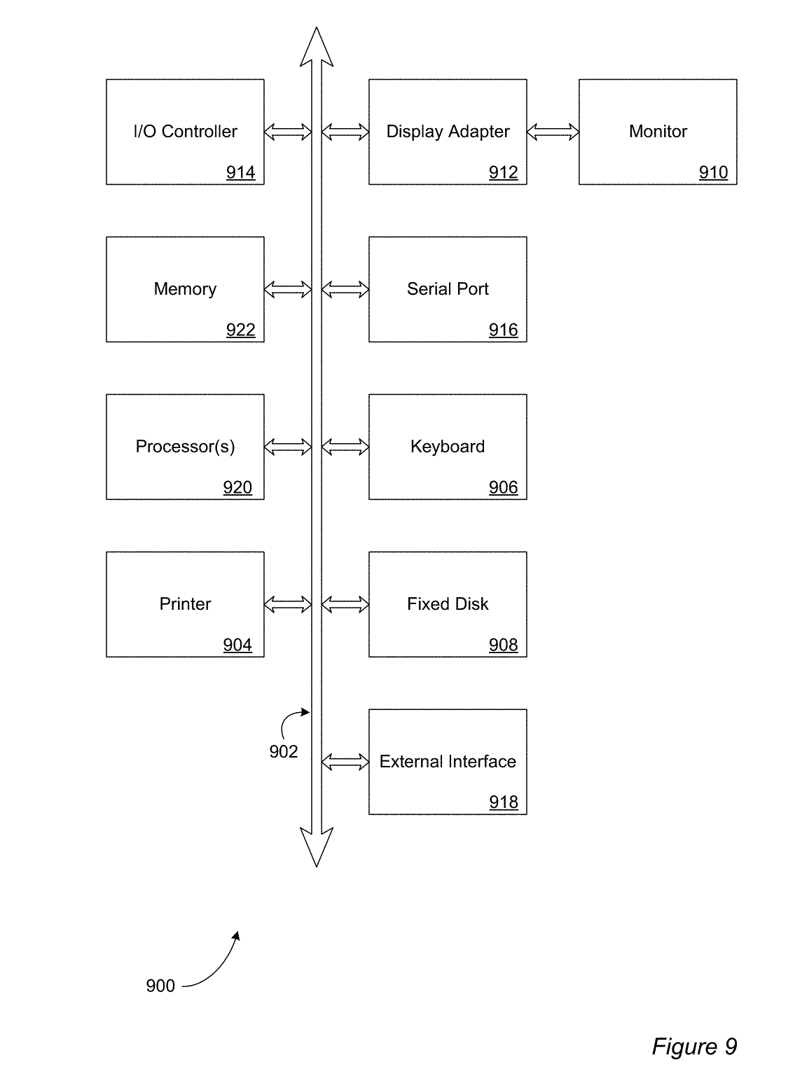

In accordance with at least one embodiment of the invention, the system, apparatus, methods, processes and/or operations described above may be wholly or partially implemented in the form of a set of instructions executed by one or more programmed computer processors such as a central processing unit (CPU) or microprocessor. Such processors may be incorporated in an apparatus, server, client or other computing device operated by, or in communication with, other components of the system. As an example, FIG. 9 depicts aspects of elements that may be present in a computing device and/or system 900 configured to implement a method and/or process in accordance with some embodiments of the present invention. The subsystems shown in FIG. 9 are interconnected via a system bus 902. Additional subsystems include a printer 904, a keyboard 906, a fixed disk 908, and a monitor 910, which is coupled to a display adapter 912. Peripherals and input/output (I/O) devices, which couple to an I/O controller 914, can be connected to the computer system with any number of means known in the art, such as a serial port 916. For example, the serial port 916 or an external interface 918 can be utilized to connect the computing device 900 to further devices and/or systems not shown in FIG. 9 including a wide area network such as the Internet, a mouse input device, and/or a scanner. The interconnection via the system bus 902 allows one or more processors 920 to communicate with each subsystem and to control the execution of instructions that may be stored in a system memory 922 and/or the fixed disk 908, as well as the exchange of information between subsystems. The system memory 922 and/or the fixed disk 908 may embody a tangible, non-transitory computer-readable medium.

All references, including publications, patent applications, and patents, cited herein are hereby incorporated by reference to the same extent as if each reference were individually and specifically indicated to be incorporated by reference and/or were set forth in its entirety herein.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely indented to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments of the invention and does not pose a limitation to the scope of the invention unless otherwise claimed. No language in the specification should be construed as indicating any non-claimed element as essential to each embodiment of the present invention.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3 and 4 and sub-ranges such as 1-3, 2-4 and 3-5, etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without clear indication to the contrary.

As used herein, the term “alternatively” refers to selection of one of two or more alternatives, and is not intended to limit the selection to only those listed alternatives or to only one of the listed alternatives at a time, unless the context clearly indicates otherwise. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Different arrangements of the components depicted in the drawings or described above, as well as components and steps not shown or described are possible. Similarly, some features and subcombinations are useful and may be employed without reference to other features and subcombinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

What is claimed is:

1. A method for nurse scheduling forecasting, comprising:
receiving, by a computer system, empirical nursing data including:
a nurse schedule for a past time interval, the nurse schedule including a number of nurses that were scheduled to work during the past time interval;
actual nurse demand during the past time interval, the actual nurse demand including a number of nurses that actually did work during the past time interval; and
a patient census during the past time interval, the patient census including a number of patients cared for by the number of nurses that actually did work during the past time interval;
training, by the computer system, a regression model with the empirical nursing data;
forecasting, by the computer system, a nurse schedule for a future time interval utilizing the trained regression model, the forecast nurse schedule including a number of nurses to be scheduled for the future time interval such that a difference between the number of nurses to be scheduled and an estimated actual nurse demand during the future time interval is optimized; and
providing, by the computer system, the forecast nurse schedule for presentation to a user.

2. A method in accordance with claim 1, wherein:
the past time interval includes a plurality of nurse work shifts; and
the empirical nursing data includes:
a number of nurses that were scheduled to work during each of the plurality of nurse work shifts;
a number of nurses that actually did work during each of the plurality of nurse work shifts; and
a patient census corresponding to each of the plurality of nurse work shifts.

3. A method in accordance with claim 1, wherein optimizing the difference between the number of nurses to be scheduled and the estimated actual nurse demand during the future time interval comprises minimizing the difference.

4. A method in accordance with claim 1, wherein the empirical nursing data further includes one or more of: type of shift, shift start time, shift end time, day of week, month of year, season, holiday indication, sporting event indication, and cultural event indication.

5. A method in accordance with claim 1, wherein forecasting the nurse schedule for the future time interval includes determining a time interval type associated with the future time interval, the time interval type corresponding to one or more of: type of shift, shift start time, shift end time, day of week, month of year, season, holiday indication, sporting event indication, and cultural event indication.

6. A method in accordance with claim 1, wherein the estimating of actual nurse demand during the future time interval is based at least in part on one or more time interval types that occur during the future time interval.

7. A method in accordance with claim 1, wherein the empirical nursing data further includes one or more additional independent variables, the one or more additional independent variables corresponding to one or more of: a number of resource nurses utilized during the past time interval, a number of nurses calling in sick during the past time interval, a number of nurses on vacation during the past time interval, a number of nurses on leave in accordance with the family medical leave act (FMLA) during the past time interval, a number of nurses cancelled by a charge nurse during the past time interval, a number of nurses working an overtime classified shift during the past time interval, a number of nurses working a premium pay shift during a past time interval, a number of nurses floated into a care unit during the past time interval, a number of nurses floated out of a care unit during the past time interval, a number of unfilled shifts during the past time interval, and a number of traveler-type nurses employed during the past time interval.

8. A method in accordance with claim 7, wherein each additional independent variable is estimated based at least in part on one or more time interval types that occur during the future time interval.

9. A method in accordance with claim 1, wherein the regression model comprises one or more of: a linear regression model, an autoregressive integrated moving average (ARIMA), a seasonal ARIMA model, a nonlinear autoregressive moving average model, an autoregressive conditional heteroskedasticity model, an autoregressive fractionally integrated moving average model, and an autoregressive moving average with exogenous inputs model.

10. A method in accordance with claim 1, wherein the future time interval has a length similar to the past time interval.

11. A method in accordance with claim 10, wherein the future time interval and the past time interval have a length of 4 to 8 weeks.

12. A method in accordance with claim 1, wherein the nurse schedule for the future time interval is constrained by a nurse recruitment process.

13. A method in accordance with claim 12, wherein the nurse recruitment process is associated with a time interval having a length of 6 to 18 months.

14. A method in accordance with claim 1, wherein the nurse schedule includes a number of nurses that were scheduled to work during the past time interval for each of a plurality of nurse types, the actual nurse demand includes a number of nurses that actually did work during the past time interval for each of the plurality of nurse types, and the patient census includes a number of patients cared for by the number of nurses that actually did work during the past time interval for each of the nurse types.

15. A method in accordance with claim 14, wherein different nurse types corresponds to different nursing skill sets.

16. A method in accordance with claim 1, wherein the nurse schedule includes a number of nurses that were scheduled to work during the past time interval for each of a plurality of nursing unit types, the actual nurse demand includes a number of nurses that actually did work during the past time interval for each of the plurality of nursing unit types, and the patient census includes a number of patients cared for by the number of nurses that actually did work during the past time interval for each of the nursing unit types.

17. A method in accordance with claim 16, wherein one or more of the nursing unit types correspond to different geographic locations.

18. A computerized system configured at least to perform the method of claim 1.

19. A computerized system for nurse schedule forecasting, the system comprising:
a data intake module configured at least to receive empirical nursing data including:
a nurse schedule for a past time interval, the nurse schedule including a number of nurses that were scheduled to work during the past time interval;
actual nurse demand during the past time interval, the actual nurse demand including a number of nurses that actually did work during the past time interval; and
a patient census during the past time interval, the patient census including a number of patients cared for by the number of nurses that actually did work during the past time interval;
a model training module configured at least to train a regression model with the empirical nursing data;
a forecasting module configured at least to forecast a nurse schedule for a future time interval utilizing the trained regression model, the forecast nurse schedule including a number of nurses to be scheduled for the future time interval such that a difference between the number of nurses to be scheduled and an estimated actual nurse demand during the future time interval is optimized; and
one or more processors configured to facilitate at least the data intake module, the model training module and the forecasting module.

20. One or more non-transitory computer-readable media collectively storing thereon computer-executable instructions that, when executed with one or more computers, perform operations comprising:
receiving empirical nursing data including:
a nurse schedule for a past time interval, the nurse schedule including a number of nurses that were scheduled to work during the past time interval;
actual nurse demand during the past time interval, the actual nurse demand including a number of nurses that actually did work during the past time interval; and
a patient census during the past time interval, the patient census including a number of patients cared for by the number of nurses that actually did work during the past time interval;
training a regression model with the empirical nursing data;
forecasting a nurse schedule for a future time interval utilizing the trained regression model, the forecast nurse schedule including a number of nurses to be scheduled for the future time interval such that a difference between the number of nurses to be scheduled and an estimated actual nurse demand during the future time interval is optimized; and
providing the forecast nurse schedule for presentation to a user.
